(19) United States
(12) Patent Application Publication
(10) Pub. No.: US 2023/0172257 A1
(43) Pub. Date: Jun. 8, 2023

(54) METHOD OF MANUFACTURING A VAPORIZABLE TABLET FOR USE IN AN ATOMIZER

(71) Applicant: Jupiter Research, LLC, Phoenix, AZ (US)

(72) Inventors: San Li, Chandler, AZ (US); Jordan Walker, Phoenix, AZ (US); Mark Scatterday, Scottsdale, AZ (US)

(21) Appl. No.: 18/052,788

(22) Filed: Nov. 4, 2022

Related U.S. Application Data
(60) Provisional application No. 63/287,126, filed on Dec. 8, 2021.
(60) Provisional application No. 63/334,989, filed on Apr. 26, 2022.

(51) Int. Cl. A24B 15/167; A24B 15/30; A24B 15/32; A24B 15/40; A24B 15/34; A61J 3/10; A61K 9/00

(52) U.S. Cl. CPC A24B 15/167; A24B 15/32; A24B 15/34; A24B 15/303; A24B 15/403; A61J 3/10; A61K 9/0073; A24F 40/20

(57) ABSTRACT

Various embodiments of the present technology may provide a method of manufacturing a vaporizable tablet for use in an atomizer of a vaporizer device. The method may include grinding a vaporizable material and treating the ground vaporizable material with a mixture comprising an aerosol forming agent and at least one binding agent. The method may also include conditioning or thermally treating the vaporizable material in a sealed enclosure or open environment for a time period. The method may further include forming the tablet utilizing the treated, conditioned vaporizable material.

### CROSS REFERENCE TO RELATED APPLICATIONS

This application is related to U.S. Provisional Pat. Application Serial No. 63/287,126, filed Dec. 8, 2021, and U.S. Provisional Pat. Application Serial No. 63/334,989, filed Apr. 26, 2022, the disclosures of which are hereby incorporated entirely herein by reference.

### BACKGROUND OF THE INVENTION

### State of the Art

“Vape” devices and heat-not-burn (HNB) devices (collectively, “vaporizer devices”) present an alternative to smoking and work by vaporizing a consumable vaporizable material by heating the vaporizable material at a lower temperature than an open flame so that a user can inhale the material in vapor form, rather than smoke.

An atomizer of a vaporizer device typically has a chamber for holding vaporizable material and a small, heated coil, in contact with the chamber. A current is typically passed through the coil, heating the chamber and the vaporizable material contained therein. Conventional forms of vaporizable material include flower, concentrates, oils, and the like. However, conventional forms of vaporizable material are ineffective at producing sufficient amounts of vapor when heated and do not efficiently release various active ingredients contained in the vaporizable material. In addition, conventional forms of vaporizable material are prone to localized heating and overheating, leaving residue in the chamber after use, thereby failing to bring out pleasant flavors and aromas of the vaporizable material.

Accordingly, what is needed is a consumable form of vaporizable material that improves sensory experiences, increases the volume of the vapor produced, improves material thermal conductivity and heating efficiency, is effective at bringing out pleasant flavors and aromas, and is easy to use with little or no advance preparation.

### SUMMARY OF THE INVENTION

Various embodiments of the present technology may provide a method of manufacturing a vaporizable tablet for use in an atomizer of a vaporizer device. The method may comprise grinding a vaporizable material and treating the ground vaporizable material with a mixture comprising an aerosol forming agent, water, and at least one binding agent. The method may also comprise conditioning or thermally treating the vaporizable material in a sealed enclosure or open environment for a time period. The method may further comprise forming the tablet utilizing the treated, conditioned vaporizable material.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures.

FIG. 1 representatively illustrates a flow diagram for manufacturing a disk-shaped tablet for use in an atomizer in accordance with an embodiment of the present technology; and

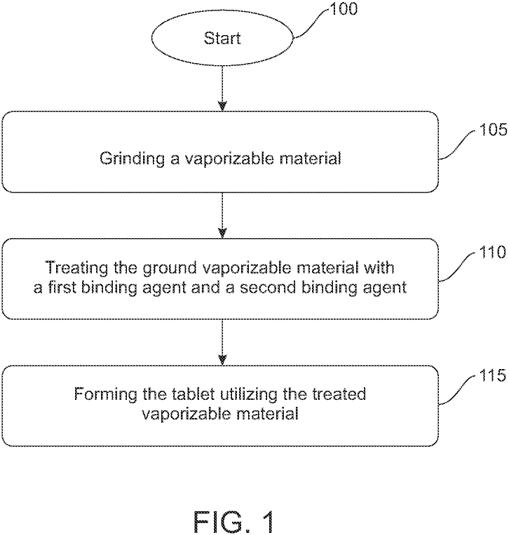

FIG. 2 representatively illustrates a flow diagram for manufacturing a spherical-shaped tablet for use in an atomizer in accordance with an embodiment of the present technology.

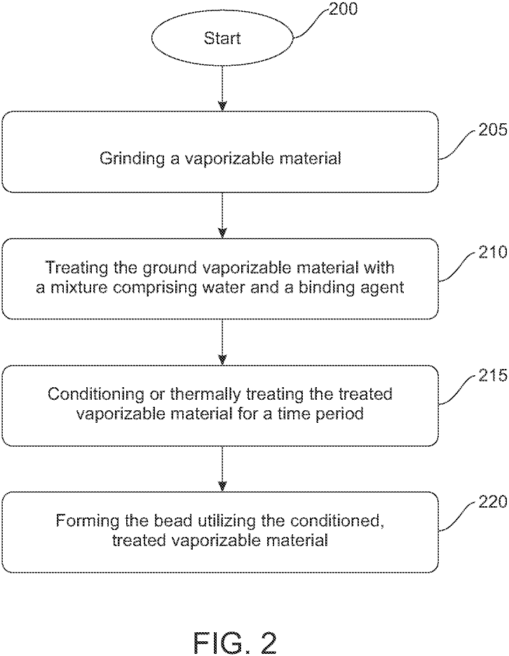

### DETAILED DESCRIPTION OF EMBODIMENTS

The present technology may be described in terms of functional components, consumables, solutions, mixtures, and materials. Such functional components, consumables, solutions, mixtures, and materials may be realized by any number of components, consumables, solutions, mixtures, and materials configured to perform the specified functions and achieve the various results. For example, the present technology may employ various atomizers, aerosol-forming agents, binding agents, chambers, heating elements, pouches, beads, mixtures, solutions, terpene mixtures, tablets, vaporizable materials, vaporizer devices, and the like, which may carry out a variety of functions. In addition, the present technology may be practiced in conjunction with any number of vaporizable materials, and the tablet described herein is merely one exemplary application for the technology.

According to various embodiments, a tablet (not shown) may be inserted into a chamber (not shown) disposed within an atomizer (not shown) of a “vape device” or heat-not-burn (HNB) device (the “vaporizer device” (not shown)). For the purposes of this Application, the term “tablet” means a capsule or a variant thereof. The tablet (not shown) may comprise any suitable shape, such as disk-shaped, spherical-shaped, oval-shaped, or triangular-shaped. The tablet (not shown) may be biodegradable and may comprise a vaporizable material, such as cannabis flower, tobacco flower, and the like, and one or more binding agents, such as a first binding agent and a second binding agent, and one or more botanic materials to modify the potency or flavor. A heating element (not shown) may be in contact with the chamber (not shown) and may heat the tablet (not shown) to a temperature sufficient to vaporize the vaporizable material. The heating element (not shown) may be suitably configured to transfer heat to the vaporizable material in any suitable manner, such as via conduction, convection, induction, and the like.

The first binding agent may comprise any suitable aerosol-forming agent to facilitate the formation of steam inside the chamber (not shown) by lowering the boiling point of the vaporizable material and to enhance the release of desired active compounds such as terpenes and cannabinoids into vapor. For example, the first binding agent may comprise vegetable glycerin (VG), propylene glycol (PG), or the like. Because the boiling point of the vaporizable material may be lowered and the thermal conducting properties improved, the vaporizable material, along with the vaporizer device (not shown), may be less prone to overheating. Accordingly, the vaporizable material may be less prone to experiencing thermal degradations, i.e., molecular deterioration as a result of overheating, thereby effectively activating various compounds within the vaporizable material to provide improved flavors and aromas.

In addition, because the first biding agent may have a higher heat capacity than the air inside the chamber (not shown), the tablet (not shown) may be effective in transmitting the heat produced by the heating element (not shown), in the form of thermal energy, to the vaporizable material contained therein. Accordingly, the vaporizer device (not shown) may conserve a greater amount of total energy, thereby allowing a user to utilize the vaporizer device (not shown) numerous times before having to change or recharge a battery (not shown) of the vaporizer device (not shown). Further, the first binding agent may be utilized to adjust a variety of active ingredient concentrations in the aerosol and/or vapor to meet a variety of sensory and user preferences. For example, in the case where the vaporizable material is cannabis flower, the first binding agent may be utilized to dilute or concentrate a variety of lipophilic, cannabinoid-derived compounds found in the cannabis flower, such as tetrahydrocannabinol (THC), cannabidiol (CBD), cannabichromene (CBC), and cannabigerol (CBG)), thereby bringing out pleasant flavors and aromas of the cannabis flower. Alternatively, in the case where the vaporizable material is tobacco flower, the first binding agent may be utilized to dilute or concentrate a variety of lipophilic, tobacco-derived compounds found in the tobacco flower, e.g., nicotine.

The second binding agent may comprise microcrystalline cellulose (MCC), starches, isomalt, or a combination thereof. Specifically, the second binding agent may be utilized to adjust various properties of the tablet (not shown), such as the porosity, hardness, or friability of the tablet (not shown).

The tablet (not shown) may comprise a diameter of about 8 mm and a thickness between about 2 and about 4 millimeters (mm) and may hold between about 80 and 120 milligrams (mg) of the vaporizable material. In one embodiment, the concentration of the first binding agent in the tablet (not shown) may be approximately 50% by weight. In an alternative embodiment, the concentration of the first binding agent in the tablet (not shown) may be less than 50% by weight. In yet another embodiment, the concentration of the first binding agent in the tablet (not shown) may greater than 50% by weight. In some embodiments, the tablet (not shown) may further comprise water (H₂O) or one or more carrier compounds, such as terpenes, or a combination thereof.

As an example, terpenes, which are generally lipophilic, meaning they have a propensity to metabolize lipophilic compounds, may aid in effectively vaporizing a variety of lipophilic compounds found within the vaporizable material. For example, in the case where the vaporizable material contained in the tablet is cannabis flower and terpenes are added to cannabis flower, the terpenes may effectively metabolize a variety of lipophilic, cannabinoid-derived compounds found in the cannabis flower, such as described in paragraph [0010] of the present Application. Further, because terpenes are generally thermally labile, meaning they break down when exposed to high temperatures, when the tablet (not shown) is heated by the heating element (not shown), the terpenes along with the lipophilic, cannabinoid-derived compounds in the cannabis flower may be broken down. Accordingly, once the vapor is produced and mixed with the air drawn into the atomizer (not shown), the resulting aerosol (combination of vapor and airflow) may be inhaled by the user so that it may effectively penetrate the deep lung region of the user. Because a high concentration of such cannabinoids may effectively penetrate the deep lung region of the user, they may be rapidly absorbed into the user’s bloodstream, thereby increasing sensations of pleasure.

It will be appreciated that modifications may be made to the tablet (not shown) without departing from the scope of the present invention. For example, instead of the disk-shaped tablet (not shown), a pouch (not shown) or one or more spherical-shaped tablets, i.e., hereinafter referred to as a bead (not shown) may be inserted into the chamber (not shown) of the vaporizer device (not shown). The pouch (not shown) may be biodegradable and/or porous and may be constructed from a variety of suitable materials, such as paper and the like.

Each bead may comprise a mixture comprising a ground vaporizable material, a binding agent, and water. As described in paragraph [0012] of this Application, the mixture may be utilized to adjust various properties of the bead, such as the porosity, hardness, friability of the bead, thermal release profiles, flavor profiles, and the like. The ground vaporizable material may comprise any suitable vaporizable material, such as hemp flower, tobacco flower, herbs, medicinal plants, and the like. In some embodiments, the mixture may further comprise one or more carrier compounds, such as terpenes, flavor compounds, and the like. Additionally, the mixture may comprise a concentrated extract of plant resin. The concentrated extract of plant resin may comprise any suitable cannabinoid.

The binding agent may comprise any suitable binding agent, such as vegetable glycerin (VG), propylene glycol (PG), microcrystalline cellulose (MCC), starch, and the like. The concentration of the binding agent and the water may be adjusted to achieve a desired boiling point of the mixture. Specifically, the ratio of the binding agent to the water may be adjusted to achieve a specific latent heat of the mixture. Because the specific latent heat of the mixture is the amount of energy that is required to change the state of a certain amount of the mixture without changing its temperature, altering the concentration of the binding agent and the water may stabilize the temperature of the bead or tablet until most of the glycerin and water is vaporized. In this regard, when the bead is heated by the heating element, the vaporizable material contained in the bead may be less prone be overheating, burning, or charring.

The boiling point temperature of the mixture may be adjusted to between about 100° C. and about 290° C. For example, a mole ratio of 9:1 of glycerin to water may produce a mixture with a boiling point temperature of approximately 210° C. The boiling point temperature may, however, be adjusted to any suitable temperature. As an example, the boiling point temperature may be lowered by increasing the concentration of water in the mixture. Because the boiling point temperature of the mixture may be lowered, the vaporizable material, along with the vaporizer device (not shown), may be less prone to overheating, thereby improving the thermal conducting properties of the vaporizable material.

The size, volume, and mass of each bead may be adjusted according to its particular application. For example, in one application, the bead may be configured to be used in a specific vaporizer device. In an alternative application, the size, volume, and mass of the bead may be chosen to dilute or concentrate a variety of active compounds of the vaporizable material. In one embodiment, the concentration of the ground vaporizable material may be about 50% by weight of the mixture, the concentration of the VG may be about 30% by weight of the mixture, and the concentration of the water and the MCC may each be about 10% by weight of the mixture. The bead may comprise any suitable diameter and weight. For example, in one embodiment, the diameter of the bead may be approximately 5 mm and the weight may be between about 100 mg and about 150 mg. In the case where the bead has a total weight of 100 mg, the bead may comprise approximately 50 mg of ground vaporizable material.

It will be appreciated that more than one bead may be vaporized at any given time and that the number of beads may be selected based on a variety of desired sensory and user preferences. For example, in the case where the vaporizable material is cannabis flower, the binding agent and the water may be utilized to dilute or concentrate a variety of lipophilic, cannabinoid-derived compounds found in the cannabis flower, such as cannabidiol (CBD). Specifically, the more beads that are vaporized at any given time, the more vapor that is produced and the stronger the sensations are for the user. As an example, one bead may produce a small amount of vapor and a light sensation (mild intensity) for the user, two beads may produce a medium amount of vapor and a medium sensation (medium intensity) for the user, and three beads may produce a large amount of vapor and a strong sensation (high intensity) for the user.

In a first operation, manufacturing the tablet (not shown) (100) may comprise grinding a vaporizable material, such as cannabis flower, tobacco flower, and the like (105). Specifically, the vaporizable material may be ground to a desired particle size. In addition, manufacturing the tablet (not shown) (100) may also comprise treating the ground vaporizable material with a first binding agent, such as vegetable glycerin (VG). After the vaporizable material is treated with the first binding agent, the vaporizable material may be further treated with the second binding agent (110). Manufacturing the tablet (not shown) (100) may further comprise forming the tablet (not shown) utilizing the treated vaporizable material (115). The tablet (not shown) may be formed using any suitable method. For example, the tablet (not shown) may be formed using the extrusion process described in U.S. Provisional Pat. Application Ser. No. 63/148,671, filed on Feb. 12, 2021. In an alternative embodiment, the tablet may be formed by compressing the treated vaporizable material in a mold to form a shape and cutting the chape to form the tablet. The tablet may comprise a uniformly shaped body.

In a second operation, manufacturing the bead (200) may comprise grinding a vaporizable material, such as hemp flower, tobacco flower, herbs, medicinal plants, and the like (205). Specifically, the vaporizable material may be ground to a desired particle size. As an example, manufacturing the bead (200) may comprise grinding 50 parts of cannabis flower by passing the 50 parts of cannabis flower through a mesh screen of any suitable size. The size of the mesh screen may be between about a 20-mesh screen and about a 100-mesh screen. For the purposes of this Application, the size of the openings and thus the size of particles captured by the screen decreases as the number indicating the mesh size increases. In addition, manufacturing the bead may comprise treating the ground cannabis flower with a mixture comprising water and a binding agent, such as vegetable glycerin (VG), propylene glycol (PG), microcrystalline cellulose (MCC), or a combination thereof (210). After the vaporizable material is treated with water and the binding agent, the treated vaporizable material may be placed in a container (not shown), sealed, and then conditioned for a specific time period, such as 24 hours (215).

Manufacturing the bead may further comprise forming the bead utilizing the treated, conditioned vaporizable material (220). The bead may be formed using any suitable method, such as by using the extrusion process described in U.S. Provisional Pat. Application Ser. No. 63/148,671, filed on Feb. 12, 2021. Specifically, the treated vaporizable material may be passed through a die to form strands of an extrudate. The strands of extrudate may then be broken down into smaller strands and then rounded to form one or more beads.

Once the tablet (not shown) or the bead is formed, it may be inserted into the chamber (not shown) of the atomizer (not shown). The user may then turn on the vaporizer device (not shown) so that the battery (not shown) may supply power to the heating element (not shown), which in turn may generate heat. Because the heating element (not shown) may be in contact with the chamber (not shown), the generated heat may be transferred to the chamber (not shown). Accordingly, the heating element (not shown) may vaporize the tablet or the bead by heating the chamber (not shown) to a temperature sufficient to generate the vapor. Once the vapor is produced, it may mix with air drawn into the atomizer (not shown) via an inlet (not shown), and the resulting aerosol (vapor and airflow) may travel as an aerosol stream along an airflow path (not shown) where it may be expelled via an outlet (not shown) and inhaled through a mouthpiece (not shown) of the vaporizer device (not shown).

The particular implementations shown and described are illustrative of the technology and its best mode and are not intended to otherwise limit the scope of the present technology in any way. Indeed, for the sake of brevity, conventional manufacturing, connection, preparation, and other functional aspects of the technology may not be described in detail.

In the foregoing description, the technology has been described with reference to specific embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any order, unless otherwise expressly specified, and are not limited to the explicit order presented in the specific examples. Additionally, the components and/or elements recited in any apparatus embodiment may be assembled or otherwise operationally configured in a variety of permutations to produce substantially the same result as the present technology and are accordingly not limited to the specific configuration recited in the specific examples.

Benefits, other advantages, and solutions to problems have been described above with regard to particular embodiments. Any benefit, advantage, solution to problems or any element that may cause any particular benefit, advantage, or solution to occur or to become more pronounced, however, is not to be construed as a critical, required, or essential feature or component.

The terms “comprises,” “comprising,” or any variation thereof, are intended to reference a non-exclusive inclusion, such that a process, method, article, composition, or apparatus that comprises a list of elements does not include only those elements recited but may also include other elements not expressly listed or inherent to such process, method, article, composition, or apparatus. Other combinations and/or modifications of the above-described structures, arrangements, applications, proportions, elements, materials, or components used in the practice of the present technology, in addition to those not specifically recited, may be varied, or otherwise particularly adapted to specific environments, manufacturing specifications, design parameters or other operating requirements without departing from the general principles of the same.

The present technology has been described above with reference to an embodiment. However, changes and modifications may be made to the embodiment without departing from the scope of the present technology. These and other changes or modifications are intended to be included within the scope of the present technology.

1. A method of manufacturing a vaporizable tablet for use in an atomizer of a vaporizer device, comprising:
grinding a vaporizable material;
treating the ground vaporizable material with a mixture comprising an aerosol-forming agent and at least one binding agent; and
forming the tablet utilizing the treated vaporizable material.

2. The method of claim 1, wherein the mixture comprises a first binding agent and a second binding agent.

3. The method of claim 1, wherein the mixture further comprises a terpene.

4. The method of claim 1, wherein the mixture further comprises a concentrated extract of plant resin comprising a cannabinoid.

5. The method of claim 1, wherein forming the tablet comprises:
compressing the treated vaporizable material in a mold to form a shape; and
cutting the shape to form the tablet, wherein the tablet comprises a uniformly shaped body.

6. The method of claim 1, wherein forming the tablet comprises:
passing the treated vaporizable material through a die to form a strand of extrudate; and
cutting the strand of extrudate to form the tablet, wherein the tablet comprises a disk-shaped body.

7. The method of claim 2, wherein the first binding agent comprises vegetable glycerin, propylene glycol, or a combination thereof.

8. The method of claim 2, wherein the second binding agent comprises microcrystalline cellulose, starches, isomalt, or a combination thereof.

9. The method of claim 3, wherein the terpene comprises at least one of bisabolol, borneol, camphene, camphor, caryophyllene, cedrene, cymene, eucalyptol, fenchol, geraniol, geranyl acetate, guaiene, guaiol, humulene, isopulegol, limonene, linalool, menthol, myrcene, nerolidol, ocimene, phellandrene, phytol, pinene, pulegone, sabinene, terpineol, terpinolene, or valencene.

10. A method of manufacturing a vaporizable tablet for use in an atomizer of a vaporizer device, comprising:
grinding a vaporizable material;
treating the ground vaporizable material with a mixture comprising water and a binding agent;
thermally conditioning the treated vaporizable material for a time period; and
forming the tablet utilizing the treated, conditioned vaporizable material.

11. The method of claim 10, wherein the binding agent comprises vegetable glycerin, propylene glycol, microcrystalline cellulose, starch, or a combination thereof.

12. The method of claim 10, wherein the mixture further comprises a terpene.

13. The method of claim 10, further comprising controlling a boiling point of the tablet by adjusting a mole ratio of the binding agent to the water.

14. The method of claim 12, wherein the terpene comprises at least one of bisabolol, borneol, camphene, camphor, caryophyllene, cedrene, cymene, eucalyptol, fenchol, geraniol, geranyl acetate, guaiene, guaiol, humulene, isopulegol, limonene, linalool, menthol, myrcene, nerolidol, ocimene, phellandrene, phytol, pinene, pulegone, sabinene, terpineol, terpinolene, or valencene.

15. The method of claim 13, wherein the boiling point is between about 100° C. and about 290° C.

16. The method of claim 10, wherein the treated vaporizable material is thermally conditioned in a sealed enclosure.

17. The method of claim 10, wherein the treated vaporizable material is thermally conditioned in an open environment.

18. The method of claim 10, wherein the time period is twenty four hours.

19. The method of claim 10, wherein forming the tablet comprises:
passing the treated, conditioned vaporizable material through a die to form a strand of extrudate;
cutting the strand of extrudate; and
rounding the strand of extrudate to form the tablet.

20. The method of claim 19, wherein the tablet comprises a spherical-shaped body, and wherein the spherical-shaped body comprises a diameter of about 8 mm and a thickness between about 2 and about 4 millimeters.
